(19) United States
(12) Patent Application Publication
(10) Pub. No.: US 2003/0204185 A1
(43) Pub. Date: Oct. 30, 2003

(54) System and method for monitoring use of disposable catheters

(76) Inventors: Marshall L. Sherman, Cardiff by the Sea, CA (US); Steven R. Katz, Northbrook, IL (US); James C. Vorisek, Riverside, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/133,019

(22) Filed: Apr. 26, 2002

(51) Int. Cl. A61B 18/18

(52) U.S. Cl. 606/41

(57) ABSTRACT

Upon initial use of a catheter, a power control system writes data indicative of the date/time of the initial use to a memory device carried by the catheter. During continued use of the catheter the power control system periodically determines the difference between the stored date/time and the current date/time. For electrophysiological catheters, the power control system allows for an electrical interface with the catheter electrodes only when the difference is less than a set time period. The electrical interface may include the provision of ablation energy from the power control system to the electrodes or the receipt of signals from the electrodes indicative of electrical activity sensed by the electrodes. Alternatively, the power control system may provide a visual or audible overtime indication when the difference is greater than the set time period.

### BACKGROUND OF THE INVENTION

1. Field of the Invention

The invention relates generally to catheter systems and, more particularly, to systems and methods for monitoring the use of disposable catheters.

2. Description of the Related Art

The heart beat in a healthy human is controlled by the sinoatrial node (“SA node”) located in the wall of the right atrium. The SA node generates electrical signal potentials that are transmitted through pathways of conductive heart tissue in the atrium to the atrioventricular node (“AV node”) which in turn transmits the electrical signals throughout the ventricle by means of the His and Purkinje conductive tissues. Improper growth, remodeling, or damage to the conductive tissue in the heart can interfere with the passage of regular electrical signals from the SA and AV nodes. Electrical signal irregularities resulting from such interference can disturb the normal rhythm of the heart and cause an abnormal rhythmic condition referred to as “cardiac arrhythmia.”

While there are different treatments for cardiac arrhythmia, including the application of anti-arrhythmia drugs, in many cases ablation of the damaged tissue can restore the correct operation of the heart. Such ablation can be performed percutaneously, i.e., a procedure in which a catheter is introduced into the patient through an artery or vein and directed to the atrium or ventricle of the heart to perform single or multiple diagnostic, therapeutic, and/or surgical procedures. In such case, an ablation procedure is used to destroy the tissue causing the arrhythmia in an attempt to remove the electrical signal irregularities or create a conductive tissue block to restore normal heart beat. Successful ablation of the conductive tissue at the arrhythmia initiation site usually terminates the arrhythmia or at least moderates the heart rhythm to acceptable levels. A widely accepted treatment for arrhythmia involves the application of RF energy to the conductive tissue.

In the case of atrial fibrillation (“AF”), a procedure published by Cox et al. and known as the “Maze procedure” involves the formation of continuous atrial incisions that prevent atrial reentry and allow sinus impulses to activate the entire myocardium. While this procedure has been found to be successful, it involves an intensely invasive approach. It is more desirable to accomplish the same result as the Maze procedure by use of a less invasive approach, such as through the use of an appropriate EP catheter system providing RF ablation therapy. In ablation therapy, transmural lesions are formed in the atria to prevent atrial reentry and to allow sinus impulses to activate the entire myocardium. In this sense transmural is meant to include lesions that pass through the atrial wall or ventricle wall from the interior surface (endocardium) to the exterior surface (epicardium).

There are two general methods of applying RF energy to cardiac tissue, unipolar and bipolar. In the unipolar method a large surface area electrode; e.g., a backplate, is placed on the chest, back or other external location of the patient to serve as a return. The backplate completes an electrical circuit with one or more electrodes that are introduced into the heart, usually via a catheter, and placed in intimate contact with the aberrant conductive tissue. In the bipolar method, electrodes introduced into the heart have different potentials and complete an electrical circuit between themselves. In both the unipolar and the bipolar methods, the current traveling between the electrodes of the catheter and between the electrodes and the backplate enters the tissue and induces a temperature rise in the tissue resulting in the creation of ablation lesions.

During ablation, RF energy is applied to the electrodes to raise the temperature of the target tissue to a lethal, non-viable state. In general, the lethal temperature boundary between viable and non-viable tissue is between approximately 45° C. to 55° C. and more specifically, approximately 48° C. Tissue heated to a temperature above 48° C. for several seconds becomes permanently non-viable and defines the ablation volume. Tissue adjacent to the electrodes delivering RF energy is heated by resistive heating which is conducted radially outward from the electrode-tissue interface. The goal is to elevate the tissue temperature, which is generally at 37° C., fairly uniformly to an ablation temperature above 48° C., while keeping both the temperature at the tissue surface and the temperature of the electrode below 100° C. In clinical applications, the target temperature is set below 70° C. to avoid coagulum formation. Lesion size has been demonstrated to be proportional to temperature.

A basic RF ablation system for forming linear lesions includes a disposable catheter carrying a plurality of electrodes, one or more patient return electrodes and a power control system adapted to provide RF power outputs to the electrodes to establish bipolar or unipolar current flow. Additional components may include a cardiac electrophysiological (“EP”) monitoring system for recording intracardiac electrograms (“ECGs”) through the catheter electrodes and a computer for viewing and logging ablation data.

Catheters used in RF ablation procedures generally include a catheter shaft with several spaced-apart band electrodes at its distal end. The electrodes transmit energy to biological tissue through conductive leads that run the length of the shaft. The distal ends of the leads pass through holes in the shaft and are electrically connected to the inside surface of the band electrode. To prevent fluid ingress into the catheter shaft through the lead holes, a hermetic seal is formed at the interface between the inside surface of the band electrodes and the outside surface of the catheter shaft. Ablation catheters also include a steering mechanism to assist in positioning of the electrodes relative to the biological site. A typical steering mechanism includes one or more steering wires attached at one end to the catheter shaft and at the other end to a steering handle. Movement of the steering handle induces tension in one or more of the steering wires which in turn causes the catheter shaft to deflect.

For reasons related to patient safety and operational integrity it is often recommended that use of a catheter be limited in some manner. For example, it is typically recommended that a catheter be used on a single patient in order to avoid the risk of cross-contamination between patients. With regard to operational integrity, repeated use of an ablation catheter beyond the recommended limit may have adverse effects. For example, excessive movement of the steering handle may cause the steering tendons to stretch or disconnect thereby reducing the physician's ability to manipulate the position of the electrodes. Furthermore, repeated torqueing of the catheter shaft and manipulations of the distal end of the catheter may disconnect the electrical leads from their respective electrodes or break the hermetic seal between the electrodes and shaft, thereby allowing fluid to enter the catheter shaft through the lead holes. Known systems for monitoring the use of catheters do so by limiting the number of times a catheter may be used for ablation. In one such system, a memory device is carried within a catheter handle. The memory device functions as a use register and is initially programmed by the manufacturer with a digital value of zero. The use register includes an input for incrementing the digital value after each application of ablation energy through the catheter's electrodes. The digital value is compared with a set value indicative of the maximum number of times a catheter may be used. If the digital value is less than the set value, continued use of the catheter is permitted. If the digital value is greater than or equal to the set value, continued use of the catheter is prevented.

A disadvantage of these known systems is their failure to account for the length of time the catheter has been used. For example, it some situations, such as the “burn and drag” procedure for creating linear lesions with a single tip electrode catheter, a large number of ablations may be performed in a short period of time. A use-monitoring system based on ablation counts, as previously described, may prevent use of a catheter if the number of ablations exceed the number allowed for that catheter. In such a situation, it maybe necessary for the physician to use multiple catheters on a single patient to complete the procedure. In other situations, a small number of ablations may be performed over a long period of time. During this time the catheter may be subjected to repeated manipulations which may effect its electrical and mechanical structural integrity. Known use-monitoring systems based on ablation counts do not account for such conditions and thus may unwittingly permit continued use of damaged catheters.

Hence, those skilled in the art have recognized a need for catheter use-monitoring systems and methods that take into consideration various procedural scenarios in which a catheter may be used. The invention fulfills these needs and others.

### SUMMARY OF THE INVENTION

Briefly, and in general terms, the invention is directed to systems and methods for monitoring the use of disposable catheters in a manner which increases patient safety and preserves catheter integrity.

In a first aspect, the invention relates to a catheter system including a catheter with a memory device and a shaft carrying at least one electrode. The system also includes a power control system having an internal clock that provides current date/time. The power control system is adapted to write data indicative of the date/time of initial use of the catheter to the memory device for storage. The power control system periodically determines the difference between the stored date/time and the current date/time and allows for an electrical interface with the at least one electrode only when the difference is less than a set time period.

By allowing for an electrical interface with the catheter electrode only when the difference between the stored date/time and the current date/time is less than a pre-set limit, the system prevents the use of catheters in a manner inconsistent with recommended practices. For example, with respect to steerable ablation catheters, in order to prevent cross-contamination between patients, it is recommended that a catheter be used only for a single patient. By limiting the time duration of catheter use, as opposed to the number of times a catheter is used, as is done in known systems, the present system makes it difficult to use a catheter in one patient, re-sterilize the catheter and then reuse the same catheter in another patient. A time based limitation on the use of a catheter also assists in maintaining the electrical and mechanical functional integrity of the catheter throughout catheter use.

In a detailed aspect, the electrical interface with the at least one electrode allows for the provision of ablation energy to the electrode and the initial use of the catheter is considered to occur when ablation energy is first provided to the at least one electrode. In another detailed facet, the electrical interface with the at least one electrode allows for the receipt of signals from the electrode indicative of electrical activity sensed by the electrode and the initial use of the catheter is considered to occur upon first receipt of an electrical signal from the electrode. In yet another detailed aspect, the power control system interfaces with the memory device through a connector and initial use of the catheter is considered to occur when the catheter is first connected to the power control system.

In another aspect, the invention relates to a catheter system including a catheter with a memory device and a power control system having an internal clock providing current time. The power control system is adapted to write data indicative of the date/time of initial use of the catheter to the memory device for storage. The power control system is also adapted to periodically determine the difference between the stored date/time and the current date/time and provide an overtime indication when the difference is greater than a set time period.

In detailed facets, the initial use of the catheter may occur in any one of the ways previously described with respect to the first aspect of the invention. In another detailed aspect, the power control system includes a visual display adapted to provide a display as the overtime indication. In yet another detailed aspect, the power control system includes a speaker adapted to produce an audible sound as the overtime indication.

In another aspect, the invention relates to a method of monitoring the use of a catheter having at least one electrode. The method includes recording the date/time of initial use of the catheter and periodically determining the elapsed time, i.e., the difference between the recorded date/time and the current date/time. The method further includes allowing for electrical interface with the at least one electrode only when the difference is less than a set time period.

In a detailed aspect, the catheter includes a memory device and recording the date/time of initial use includes writing data indicative of the date/time to the memory device. In a further detailed aspect, the method includes checking the memory device for previously recorded date/time data and if no previously recorded date/time data is present, writing data indicative of the date/time to the memory device. If previously recorded date/time data is present, the method includes proceeding with periodically determining the elapsed time and allowing for electrical interface accordingly. In another detailed aspect, the memory device is provided with data indicative of an expiration date of the catheter and the method includes comparing the expiration date to the current date/time and, if the current date/time is less than the expiration date, proceeding with recording the date/time of initial use of the catheter, periodically determining the elapsed time and allowing for electrical interface.

These and other aspects and advantages of the invention will become apparent from the following detailed description and the accompanying drawings which illustrate by way of example the features of the invention.

### BRIEF DESCRIPTION OF THE DRAWINGS

FIG. 1 is a schematic block diagram of an ablation system including a power control system, two patient return electrodes, a catheter system, a computer and an electrophysiological (“EP”) monitoring system;

FIG. 2 is a diagram of the catheter system of FIG. 1 presenting more detail including a handle and a catheter shaft having a preformed distal segment carrying a linear array of electrodes;

FIG. 3 is a detailed block diagram of a portion of the distal segment of the catheter system of FIG. 2, depicting a tip electrode and several band electrodes;

FIG. 4 is a schematic block diagram of a memory device and surge suppressor carried by the catheter handle and isolation circuitry within the and forming the power control system, the combination of which forms a use-monitoring system;

FIG. 5 is the schematic block diagram of FIG. 4 with an alternate memory device;

FIG. 6 is a schematic diagram of the isolation circuitry of FIGS. 4 and 5; and

FIG. 7 is a flowchart of the operation of the use-monitoring system.

### DETAILED DESCRIPTION OF THE PREFERRED EMBODIMENTS

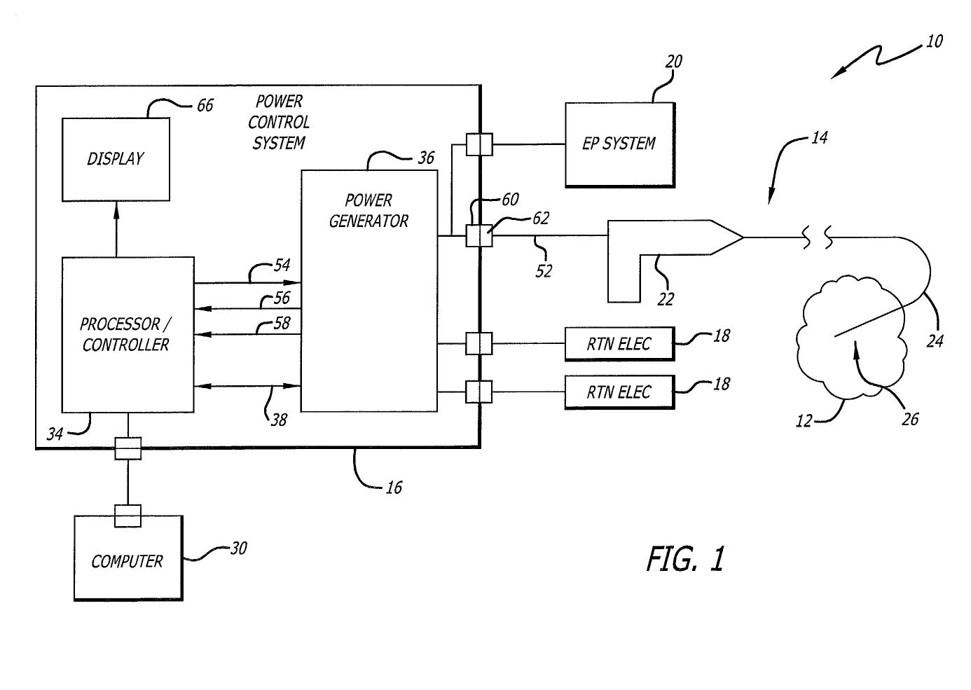

Turning now to the drawings, in which like reference numerals are used to designate like or corresponding elements among the several figures, in FIG. 1 there is shown an ablation system 10 for use in ablation therapy of a biological site 12, e.g., the atrium or ventricle of the heart. The ablation system 10 includes a catheter system 14, a power control system 16, a pair of patient return electrodes 18, an EP monitoring system 20 and a computer 30. The catheter system 14 provides RF current supplied by the power control system 16 to the biological site 12. The patient return electrodes 18 provide the return path for the RF current. The EP monitoring system 20 collects and displays electrograms from within the biological site through the electrodes at the distal end of the catheter, while the computer 30 displays, collects and logs ablation data.

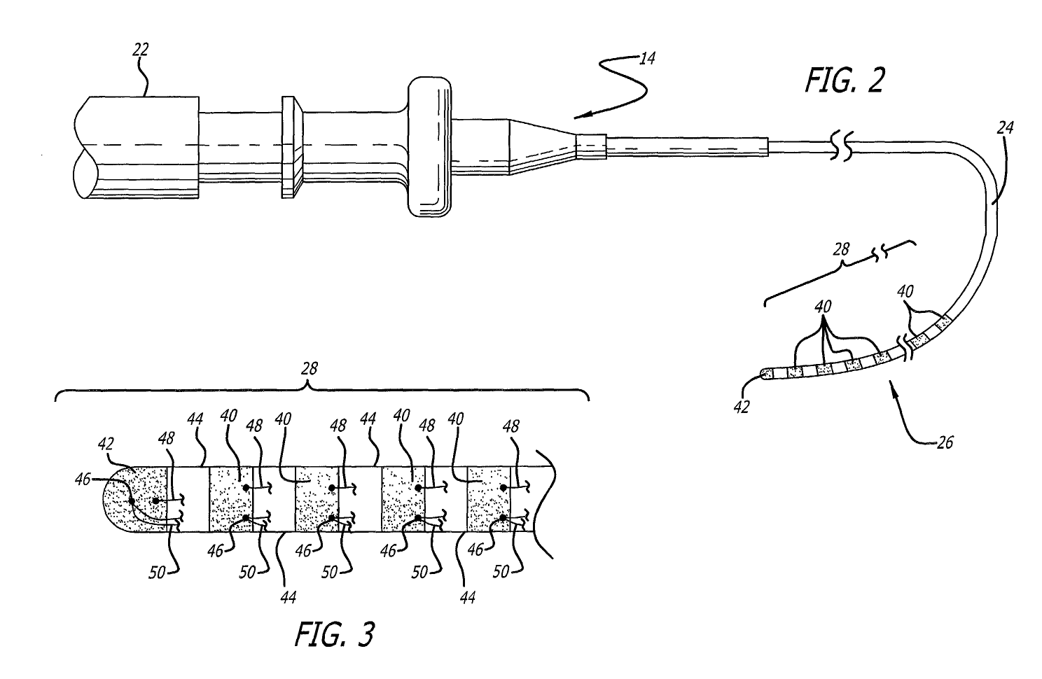

The catheter system 14 includes a handle 22 and a steerable catheter shaft 24 having a distal segment 26 that is capable of being percutaneously introduced into the biological site 12. As shown in FIGS. 2 and 3, the distal segment 26 of the catheter system 14 includes an electrode system 28. The electrode system 28 is shown in schematic form with the components drawn in more detail to more clearly illustrate the relationship between the components. A preferred embodiment of the electrode system 28 includes twelve band electrodes 40 arranged in a substantially linear array along the distal segment 26 of the catheter shaft 24. The electrode system 28 may include a tip electrode 42. (For clarity of illustration, only six band electrodes 40 are shown in FIG. 2 and only four band electrodes 40 are shown in FIG. 3 although as stated, a preferred embodiment may include many more.) The band electrodes 40 are arranged so that there is an electrically non-conductive space 44 between adjacent electrodes. In one configuration of the electrode system 28, the width of the band electrodes 40 is 3 mm and the space 44 between the electrodes is 4 mm. The total length of the electrode system 28, as such, is approximately 8 cm.

The band electrodes 40 are formed of a material having a significantly higher thermal conductivity than that of the biological tissue to be ablated. Possible materials include silver, gold, chromium, aluminum, molybdenum, tungsten, nickel, platinum, and platinum/10% iridium. Because of the difference in thermal conductivity between the band electrodes 40 and the tissue, the electrodes cool off more rapidly in the flowing fluids at the biological site. The band electrodes 40 are sized so that the surface area available for contact with fluid in the heart, e.g., blood, is sufficient to allow for efficient heat dissipation from the electrodes to the surrounding blood. In a preferred embodiment, the band electrodes 40 are 7 French (2.3 mm in diameter) with a length of 3 mm and a thickness in the range of about 0.002 mm to about 0.010 mm.

Associated with the electrode system 28 are thermal sensors 46 for monitoring the temperature of the electrode system 28 at various points along its length. In one embodiment, each electrode 40, 42 has a thermal sensor 46 mounted to it. In another embodiment of the electrode system 28 a thermal sensor 46 is mounted on every other band electrode 40. Thus for a catheter having twelve electrodes, there are thermal sensors on six electrodes. In yet another embodiment of the electrode system 28 the odd numbered electrodes have one thermal sensor 46 while the even numbered electrodes have two thermal sensors. In yet another embodiment of the electrode system 28 the electrodes have two thermal sensors 46. In FIG. 3, which shows an embodiment having one thermal sensor for each electrode, there is shown a single drive wire 48 for each electrode 40 to provide power to each electrode for ablation purposes and two temperature leads 50 for each thermal sensor 46 to establish a thermocouple effect. In another configuration (not shown), the power lead acts as one of the thermocouple leads thereby reducing the number of wires. Details of such configurations are disclosed in U.S. Pat. Nos. 6,042,580, 6,045,550 and 6,049,737 the disclosures of which are hereby incorporated by reference. In alternate embodiments, the thermal sensors 46 may include thermistors, RTDs and fluoroptic probes. The power leads 48 and thermocouple leads 50 travel the length of the shaft 24 and through the handle 22 to interface with the power control system 16 through a catheter receptacle 60 (FIG. 1).

With reference to FIG. 1, the power control system 14 includes a power generator 36, that may have any number of output channels through which it provides power to the electrodes of the electrode system 28. The operation of the power generator 36 is controlled by a processor/controller 34 which outputs control signals 54 to the power generator 36. The processor/controller 34 monitors the power provided by the power generator 36 over a power monitor line 56. In addition, the processor/controller 34 also monitors the temperatures of the electrodes within the electrode system 28 over a temperature monitor line 58. Based on the monitored power and temperature, the processor/controller 34 adjusts the operation of the power generator 36 and thus the power provided to the electrodes, either collectively in groups or zones, or individually. Details of a multichannel power control system are disclosed in U.S. Pat. Nos. 6,050,994, 6,059,778 and 6,171,305, the disclosures of which are hereby incorporated by reference.

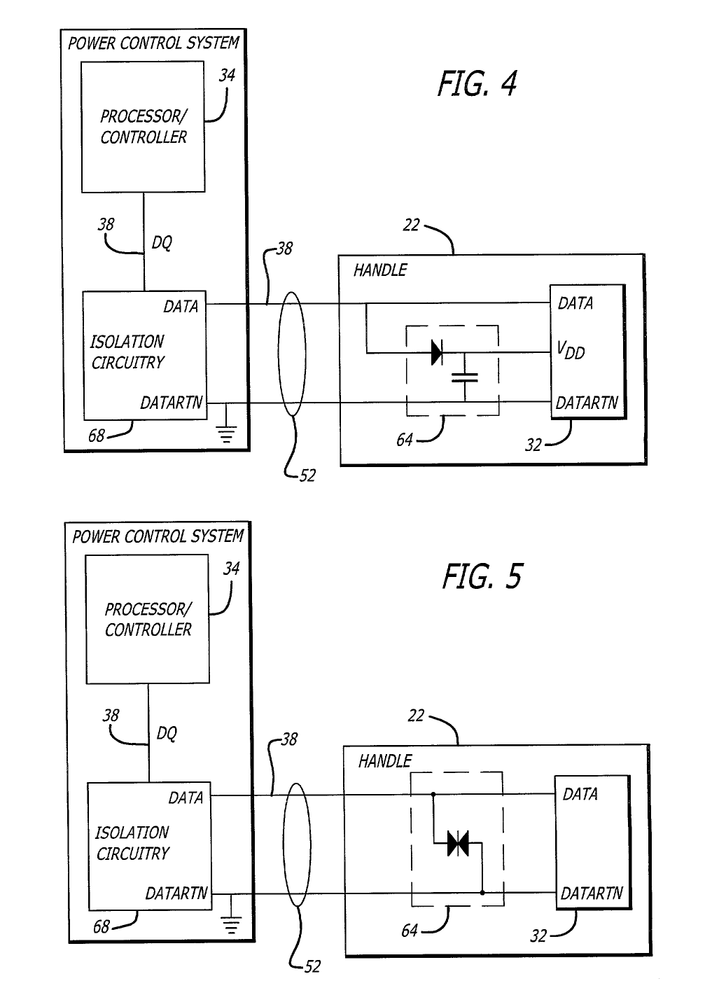

With reference to FIG. 4, a memory device 32, such as a Dallas Semiconductor DS1820, is located in the catheter handle 22. In one embodiment, the memory device 32 is provided with a portion, i.e., page, of blank memory. Upon initial use of the catheter 14 data indicative of the date/time of initial use is written to the memory device 32 from the processor/controller 34 and the page is then write protected to prevent the date/time of initial use from being modified. “Initial use”, as used herein may mean the first time a catheter 14 is recognized by the power control system 16 as being physically connected thereto. Catheter recognition may occur by confirming the presence of thermocouples such as is done during execution of an ablation system set-up algorithm described further below. Alternatively, an initial use may mean the first time a catheter 14 is connected to a power control system 16 and ablation energy is transmitted to the catheter's electrode system 28 or electrical signals sensed by the electrodes are received by the power control system. After initial use of the catheter 14, the processor/controller 34 monitors the elapsed time since initial use and controls future use of the catheter accordingly, as described in detail below.

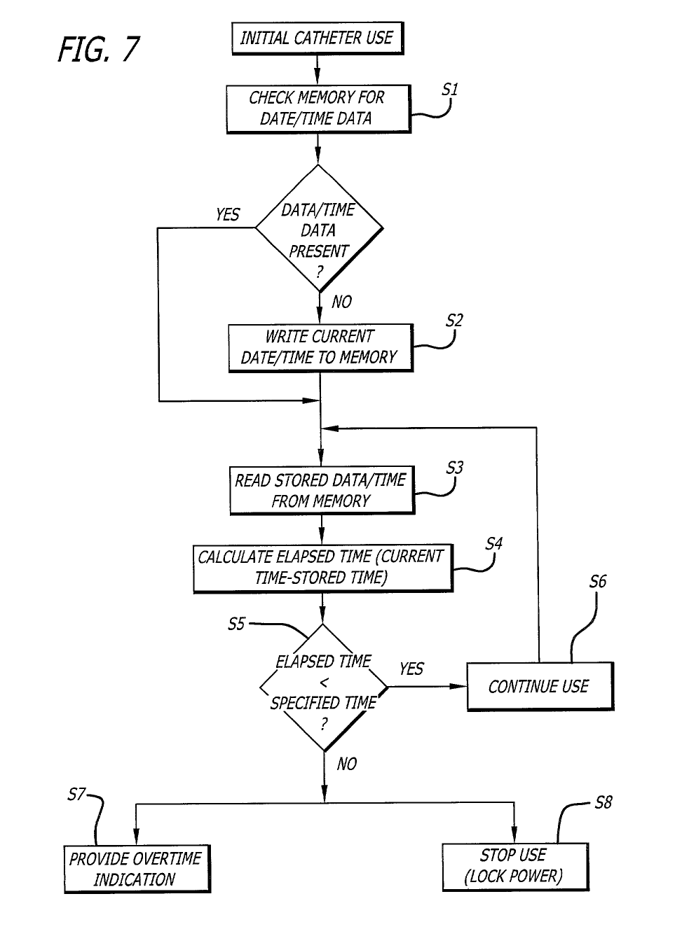

With continued reference to FIG. 4, both the power input V_{DD} and the data port DQ of the memory device 32 are connected to the processor/controller 34 (FIG. 1), by way of a signal line 38 passing through isolation circuitry 68 within the power generator 36 and through the catheter cable 52. Packets of data, e.g., read and write commands, are sent to both the power input V_{DD} and the data port DQ by the processor/controller 34 as part of the use-monitoring process. The DQ line is used by the processor/controller 34 to first record, and later read, the date/time data to/from the memory device 32. Details of this operation are provided below with reference top FIG. 7.

The digital signals within the packets of data are sufficient to power the memory device 32 through the power input V_{DD}. The memory device 32 includes a capacitor (not shown) that stores energy when the signal line 38 to the V_{DD} input is high, i.e., during read commands. When the signal line 38 is low, the memory device 32 operates off of the energy stored in the capacitor. Powering the memory device 32 in this manner avoids the application of a DC signal to the device As such, the possibility of a short between a DC signal and an electrode lead wire in the catheter cable 52, and the potential for a cardiac arrthymia induced thereby, is eliminated.

The handle 22 also includes a voltage suppressor 64, such as a ON Semiconductor mini-MOSORB 12V bi-directional Zener transient voltage suppressor, part number SA12CA. The voltage suppressor 64 protects the memory device 32 from large voltages, such as those resulting from electrostatic discharge through the catheter cable 52. The voltage suppressor 64 behaves as a high impedance resistor during normal operating voltages, e.g., about 5 volts, but serves as a shunt if significantly higher voltages occur, e.g., above approximately 10 volts.

With reference to FIG. 5, in an alternate embodiment, the memory device 32 comprises a Dallas Semiconductor DS2502. In this configuration the memory device 32 receives data through and is powered through a single data port DQ. Details of the operation of this configuration are similar to those described with reference to FIG. 4.

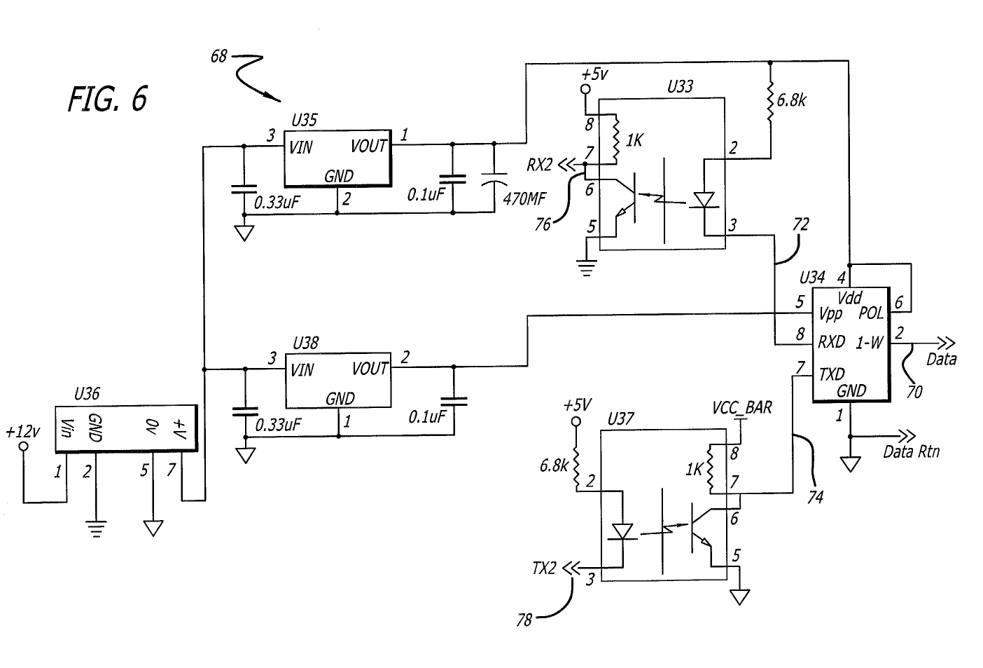

With reference to FIG. 6, the isolation circuitry 68 within the power generator includes various circuit components which function to isolate the memory device 32 from the power generator electronics. This isolation prevents leakage currents and voltage potentials from reaching the patient and thus provides for increased patient safety. The signals which are isolated include: ground, +12V, RX2 (receive serial channel 2) and TX2 (transmit serial channel 2).

The memory device 32 in the handle and the serial interface U34 are serial devices that communicate with each other over data line 70. The serial interface U34 receives data streams from the memory device 32 on pin 2 with a reference to patient ground on pin 1. The serial interface U34, in turn, communicates with a pair of optoisolators U33, U37 over receive line 72 and transmit line 74, respectively and receives signals only after they have been optically isolated. The optoisolators U33, U37 communicate with the processor/controller 34 through receive output 76 and transmit output 78 at a baud rate of 9400.

Referring to FIGS. 3, 4, 5 and 6, the following devices are shown. All resistors are 1% unless otherwise noted.

| Device | Part No. | Manufacturer |
|---|---|---|
| U33 | HPCL-2300 | Hewlett-Packard |
| U34 | DS2480 | Dallas Semiconductor |
| U35 | L78L05 | ST Microelectronics |
| U36 | NMV1215SA | Newport Component |
| U37 | HPCL-2300 | Hewlett-Packard |
| U38 | LT1086CT-12 | Linear Tech |

With reference to FIG. 1, during ablation system set up, the processor/controller 34 initiates a set-up algorithm that verifies both the presence of and the integrity of the catheter. For a catheter configured such that the power lead acts as one of the thermocouple leads as disclosed in U.S. Pat. Nos. 6,042,580, 6,045,550 and 6,049,737, the catheter integrity check includes testing of the drive wire 48 and thermocouple lead 50 connection between the catheter electrodes 40, 42 and the pin connector 62 at the proximal end of the catheter system 14.

The electrode system 28 is maintained at a temperature different then that of the handle, where a cold junction reference temperature is measured. The temperature difference forces each thermocouple 46 to output a voltage indicative of the temperature at the thermocouple. This voltage is output by the catheter and received by the processor/controller 34 over the temperature monitor line 58. The set-up algorithm verifies the presence of a voltage for each thermocouple 46. The absence of an output voltage for any particular thermocouple 46 is indicative of a faulty wire or lead 48, 50 connection for that thermocouple. Because one of the leads for the thermocouple 46 functions as a drive wire 48, the output of a voltage from a thermocouple also confirms proper power lead connection.

In addition to the integrity check just described, the processor/controller 34 also, upon confirmation of the presence of a catheter, executes a use-monitoring algorithm which monitors the use of the catheter 14 to prevent use of the catheter beyond a specified period of time. In one configuration, the specified period of time is twelve hours. This time, however, is not critical to the operation of the use-monitoring system and any other time may be used depending on the particular catheter. The period of time may be programmed into the processor/controller 34 through computer 30 or through front panel controls on the power control system 16. It may also be stored in the catheter's memory device 32, in which case the processor/controller 34 reads the specified time period from the memory device.

The operation of the use-monitoring algorithm is described with reference to FIG. 7. In general, FIG. 7 shows a flowchart that sets forth not only the operation of the use-monitoring algorithm, but also the architecture and functionality of the use-monitoring system. The blocks of the flowchart can be viewed as sections of software code. In a preferred embodiment the software code is stored in the processor/controller 34. A detailed description of one configuration of the use-monitoring code, i.e., reuse code, is provided in the Appendix, the contents of which is hereby incorporated.

With reference to FIG. 7, upon initial use of the catheter the processor/controller, at step S1, checks the memory device for previously stored date/time data. If date/time data is not stored in the memory device, the processor/controller, at step S2, writes the current date/time to the memory. The current date/time is based on the internal clock of the processor/controller.

If date/time data is stored in the memory device, the processor/controller, at steps S3 and S4, reads the stored date/time from the memory device and calculates the difference between it and the current date/time of its internal clock to determine the time that has elapsed since the initial use of the catheter. At step S5, the processor/controller compares the elapsed time to the specified time period of use associated with the catheter. If the elapsed time is less than the time period, use of the catheter continues at step S6 and the monitoring process continues at step S3.

If the elapsed time is equal to or exceeds the specified time period of use, an overtime indication is provided at step S7 and/or continued use of the catheter is prohibited at step S8. Catheter use may be prohibited by preventing power from being output from the power generator. This maybe done, for example, using controllable relay interconnects at the outputs of the power generator, such as shown in U.S. Pat. No. 6,042,580. The processor/controller outputs control signals to open the relay interconnects, thereby preventing the output of power. The overtime indication may include a visual display on the display 66 (FIG. 1) of the power control system. It may also include an audible indication over a speaker (not shown). In either instance, the processor/controller outputs signals to the display and/or speaker that cause an appropriate indication, e.g., “catheter time exceeded, discontinue use”, to occur.

The memory device 32 is nonvolatile. Accordingly, the catheter 14 may be disconnected from one power control system 16 and reconnected to another power control system without losing the date/time of initial use.

Some catheters have an expiration date after which they should not be used. In another embodiment, the memory device 32 maybe programmed to contain the expiration date for the catheter. Upon connection of the catheter to power control system 16, the processor/controller 34 reads the expiration date and compares it with the current date. If the current date is beyond the expiration date, use of the catheter is prohibited. If the current date is earlier than the expiration date, the expiration date is overwritten with the current date/time and use of the catheter is allowed to proceed in accordance with FIG. 5.

The system and method thus described maybe easily adapted for use in other ablation systems and is in no way limited to cardiac RF ablation systems. The system and method may find adaptation in any ablation system in which a power control system interfaces with a catheter. Other examples of ablation energy sources for power control systems are ultrasound, laser, microwave and cyrogenic energy, each of which results in the ablation of biological tissue.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A catheter system comprising:
a catheter with a memory device and a shaft carrying at least one electrode; and
a power control system having an internal clock providing current date/time, the power control system adapted to:
(a) write data indicative of the date/time of initial use of the catheter to the memory device for storage therein;
(b) periodically determine the difference between the stored date/time and the current date/time; and
(c) allow for an electrical interface with the at least one electrode only when the difference is less than a set time period.

2. The catheter system of claim 1 wherein the electrical interface with the at least one electrode allows for the provision of ablation energy to the electrode.

3. The catheter system of claim 2 wherein the initial use of the catheter is considered to occur when ablation energy is first provided to the at least one electrode.

4. The catheter system of claim 1 wherein the electrical interface with the at least one electrode allows for the receipt of signals from the electrode indicative of electrical activity sensed by the electrode.

5. The catheter system of claim 4 wherein the initial use of the catheter is considered to occur upon first receipt of an electrical signal from the electrode.

6. The catheter system of claim 1 wherein the power control system interfaces with the memory device through a connector and initial use of the catheter is considered to occur when the catheter is first connected to the power control system.

7. The catheter system of claim 1 wherein the memory device comprises a memory page, the data indicative of the date/time of initial use is stored in the memory page and the memory page is write protected after having the data stored therein.

8. The catheter system of claim 1 wherein the memory device is provided with data indicative of an expiration date of the catheter and the power control system is further adapted to:
compare the expiration date to the current date/time; and
proceed with adaptations (a), (b) and (c) only if the current date/time is prior to the expiration date.

9. The catheter system of claim 1 wherein the memory device is provided with blank memory and the power control system is further adapted to:
check the memory device for memorized data; and
proceed with adaptation (a) when the memory device is blank, otherwise proceed with adaptations (b) and (c).

10. The catheter system of claim 1 wherein the memory device is adapted to be powered by a digital data source within the power control system.

11. The catheter system of claim 10 further comprising a voltage suppressor positioned between the digital data source and the memory device.

12. The catheter system of claim 1 wherein the power control system comprises:
a power generator for providing RF power to the at least one electrode; and
an isolation circuit for electrically isolating the power generator from the memory device.

13. A catheter system comprising:
a catheter with a memory device; and
a power control system having an internal clock providing current time, the power control system adapted to:
(a) write data indicative of the date/time of initial use of the catheter to the memory device for storage therein;
(b) periodically determine the difference between the stored date/time and the current date/time; and
(c) provide an overtime indication when the difference is greater than a set time period.

14. The catheter system of claim 13 wherein the catheter further comprises an energy delivery system, the power control system is adapted to provide energy to the energy delivery system and initial use of the catheter is considered to occur when energy is first provided to the energy delivery system.

15. The catheter system of claim 13 wherein the memory device is provided with data indicative of an expiration date of the catheter and the power control system is further adapted to:
compare the expiration date to the current date/time; and
proceed with adaptations (a), (b) and (c) only if the current date/time is prior to the expiration date.

16. The catheter system of claim 13 wherein the memory device is provided with blank memory and the power control system is further adapted to:
check the memory device for memorized data; and
proceed with adaptation (a) when the memory device is blank, otherwise proceed with adaptations (b) and (c).

17. The catheter system of claim 13 further comprising an energy delivery system carried by the catheter, wherein the power control system interfaces with the energy delivery system and is further adapted to provide energy to the energy delivery system only when the difference is less than the set time period.

18. A method of monitoring the use of a catheter having at least one electrode, said method comprising:
(a) recording the date/time of initial use of the catheter;
(b) periodically determining the difference between the recorded date/time and the current date/time; and
(c) allowing for electrical interface with the at least one electrode only when the difference is less than a set time period.

19. The method of claim 18 wherein the catheter includes a memory device and recording the date/time of initial use comprises writing data indicative of the date/time to the memory device.

20. The method of claim 19 wherein the memory device comprises a memory page, the data indicative of the date/time of initial use is stored in the memory page and recording the date/time of initial use further comprises write protecting the memory page after the data is stored therein.

21. The method of claim 19 further comprising:
checking the memory device for previously recorded date/time data;
if no previously recorded date/time data is present, writing data indicative of the date/time to the memory device; and
if previously recorded date/time data is present, proceeding with (b) and (c).

22. The method of claim 19 wherein the memory device is provided with data indicative of an expiration date of the catheter and further comprising:
comparing the expiration date to the current date/time; and
proceeding with (a), (b) and (c) only if the current date/time is less than the expiration date.

23. The method of claim 18 wherein allowing for electrical interface with the at least one electrode comprises providing ablation energy to the electrode.

24. The method of claim 23 wherein the initial use of the catheter is considered to occur when ablation energy is first provided to the at least one electrode.

25. The method of claim 18 wherein allowing for electrical interface with the at least one electrode comprises receiving signals from the electrode indicative of electrical activity sensed by the electrode.

26. The method of claim 25 wherein the initial use of the catheter is considered to occur upon first receipt of an electrical signal from the electrode.
